(19) United States
(12) Patent Application Publication
(10) Pub. No.: US 2011/0281944 A1
(43) Pub. Date: Nov. 17, 2011

(54) METHOD OF A RHEIN COMPOUND FOR INHIBITING PANCREATIC ISLET BETA-CELL DYSFUNCTION AND PREVENTING OR TREATING A PANCREATIC ISLET BETA-CELL DYSFUNCTION RELATED DISORDER

(75) Inventor: Zhihong Liu, Nanjing (CN)

(73) Assignee: Research Institute of Nephrology of Nanjing PLA, China, Nanjing (CN)

(21) Appl. No.: 12/906,817

(22) Filed: Oct. 18, 2010

(30) Foreign Application Priority Data
May 13, 2010 (CN) 201010171074.3

(51) Int. Cl. A61K 31/235; A61P 3/04; A61P 3/10; A61K 31/192; A61P 1/18

(52) U.S. Cl. 514/510; 514/569

(57) ABSTRACT

This invention provides a method for inhibiting pancreatic islet β-cell dysfunction, comprising administering to a subject in need thereof an inhibitory effective amount of a rhein compound having the general formula (I) or a pharmaceutically acceptable salt thereof,

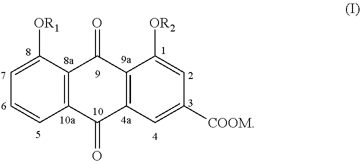

### CROSS-REFERENCE TO RELATED APPLICATIONS

This non-provisional application claims priority under 35 U.S.C. §119(a) on Patent Application No(s). 201010171074.3 filed in China on May 13, 2010, the entire contents of which are hereby incorporated by reference.

### BACKGROUND OF THE INVENTION

1. Technical Field

The present invention relates to a method for inhibiting pancreatic islet β-cell dysfunction and a method for preventing or treating a pancreatic islet β-cell dysfunction related disorder by use of a rhein compound or a pharmaceutically acceptable salt thereof.

2. Background Art

In humans, the function of pancreatic islet β-cell undergoes a series of changes during the development from obesity to insulin resistance, and finally to type 2 diabetes. In the initial stages, β-cells can secret sufficient insulin as a compensation so as to maintain the blood sugar at a normal level. However, long-term over-secretion of insulin would result in function decompensation and failure of islet cells, and ultimately lead to the occurrence and development of type 2 diabetes. The results from United Kingdom Prospective Diabetes Study (UKPDS) show that the β-cell function of the newly-diagnosed patients suffering from type 2 diabetes only is about 50% that of the normal persons, and declines at a rate of 4.5% per year. At present, clinically there still lacks effective treatments for protecting and repairing pancreatic islet β-cell function.

Rhein compounds or salts thereof are compounds with established structure as follows,

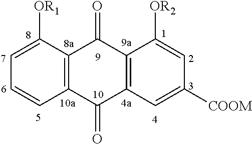

wherein M is H, alkali metal, alkaline-earth metal or organic base residue, and R₁ and R₂ are independently of each other H or acetyl group.

Presently, the representative examples of rhein compounds or salts thereof are rhein (each of M, R₁ and R₂ is H), sodium rhein (M is Na, and both R₁ and R₂ are H), potassium rhein (M is K, and both R₁ and R₂ are H), 1,8-diacetyl rhein (M is H, and both R₁ and R₂ are acetyl group), 1,8-diacetyl rhein sodium (M is Na, and R₁ and R₂ are acetyl group) and 1,8-diacetyl rhein potassium (M is K, and R₁ and R₂=acetyl group). In intestines, the acetyl group of 1,8-diacetyl rhein may be completely hydrolyzed, and the active form is rhein.

### SUMMARY OF THE INVENTION

The present invention provides a method for inhibiting pancreatic islet β-cell dysfunction, comprising administering to a subject in need thereof an inhibitory effective amount of a rhein compound having the general formula (I) or a pharmaceutically acceptable salt thereof,

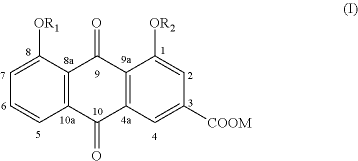

wherein M is H, alkali metal, alkaline-earth metal or organic base residue, and R₁ and R₂ are independently selected from H and acetyl.

In one preferred embodiment according to the method of the present invention, in the general formula above, both R₁ and R₂ are H, or both R₁ and R₂ are acetyl.

In one preferred embodiment according to the method of the present invention, in the general formula above, M is H or alkali metal.

In this invention, a rhein compound having the general formula (I) or a pharmaceutically acceptable salt thereof can be administered for inhibiting pancreatic islet β-cell dysfunction at an inhibitory effective amount of 35˜140 mg/kg, preferably at an inhibitory effective amount of 120 mg/kg.

The present invention provides a method for preventing or treating a pancreatic islet β-cell dysfunction related disorder, comprising administering to a subject in need thereof a therapeutic effective amount of a rhein compound or a pharmaceutically acceptable salt thereof above mentioned.

In this invention, a subject can be any mammal including a human. In particular embodiments, the subject is a human.

In this invention, a rhein compound having the general formula (I) or a pharmaceutically acceptable salt thereof can be administered for preventing or treating a pancreatic islet β-cell dysfunction related disorder at a therapeutic effective amount of 35˜140 mg/kg, preferably at a therapeutic effective amount of 120 mg/kg.

In this invention, a pancreatic islet β-cell dysfunction related disorder includes metabolic syndromes, such as obesity and diabetes mellitus, and the diabetes mellitus are particularly type 2 diabetes.

The inventor found that the rhein compound or the pharmaceutically acceptable salt thereof in this invention could protect and repair the functions of pancreatic islet β-cells, and could inhibit pancreatic islet β-cells dysfunction for patients suffering from metabolic syndromes, such as obesity and diabetes mellitus. Thus, the rhein compound or a pharmaceutically acceptable salt thereof can be used for preventing and treating a pancreatic islet β-cells dysfunction related disorder such as a diabete, particular a type 2 diabete.

### BRIEF DESCRIPTION OF THE DRAWINGS

FIG. 1 shows IPGTT results of rhein-treated group and control group.

FIG. 2 shows pancreatic islet perfusion with rhein Ex vivo significantly improves the first phase insulin secretion in type 2 diabetic db/db mice.

FIG. 3 shows rhein intervention increases the mass of pancreatic islet β-cells of db/db mice, *p<0.05, as compared with db/db mice.

FIG. 4 shows insulin staining of rhein-treated group and control group.

### DETAILED DESCRIPTION OF THE INVENTION

The characteristics and advantages of this invention can be understood well by the illustration of the examples below. However, this invention is not limited to these examples.

The studies of the present invention show that oral administration of rhein can very significantly improve the glucose tolerance of obese insulin-resistant rat and type 2 diabetic db/db mice, reduce the loss of pancreatic islet β-cells, and protect the function of pancreatic islet β-cell. The present inventors find and confirm that rhein has the protective effect on pancreatic islet β-cell, and can be applied into the treatment of metabolic syndromes, such as obesity and diabetes mellitus.

According to the experiments of the present invention, it is shown that rhein improves the function of pancreatic islet β-cells in the obese rats, and increased the content of pancreatic islet β-cells. The hyperglycemic clamp experiment shows that the glucose infusion rate (GIR) at steady-state in the rhein-treated group [(26.1±2.9) mg.kg⁻¹ min⁻¹] is significantly higher than that in the untreated obese group [(35.9±4.1) mg.kg⁻¹.min⁻¹, P<0.05] after treatment with rhein for 4 weeks (Table 1). In the normal control group, the insulin is strongly strained and uniformly distributed in the pancreatic islets. The insulin expression level in the obese control group is merely 70% that in the normal control group. Compared with the rats in the obese control group, rhein treatment significantly improves the insulin expression level in the islets.

TABLE 1
Hyperglycemic clamp characteristic parameters in obese rats after
treatment with rhein for 4 weeks

| | Normal control | Obese | rhein-treated obese |
|---|---|---|---|
| Parameters | group | group | group |
| FBG (mmol/L) | 6.2 ± 0.5 | 6.9 ± 0.6 | 5.3 ± 0.5 |
| SSBG(mmol/L) | 14.9 ± 0.8 | 15.2 ± 0.7 | 14.9 ± 0.8 |
| GIR(mg.kg⁻¹ .min⁻¹) | 35.7 ± 3.3 | 23.1 ± 3.2* | 32.2 ± 2.9# |

*P < 0.05 vs normal control,
#P < 0.05 vs Obese group;
FBG: Fasting blood glucose;
SSBG: Steady state blood glucose;
GIR: Glucose infusion rate

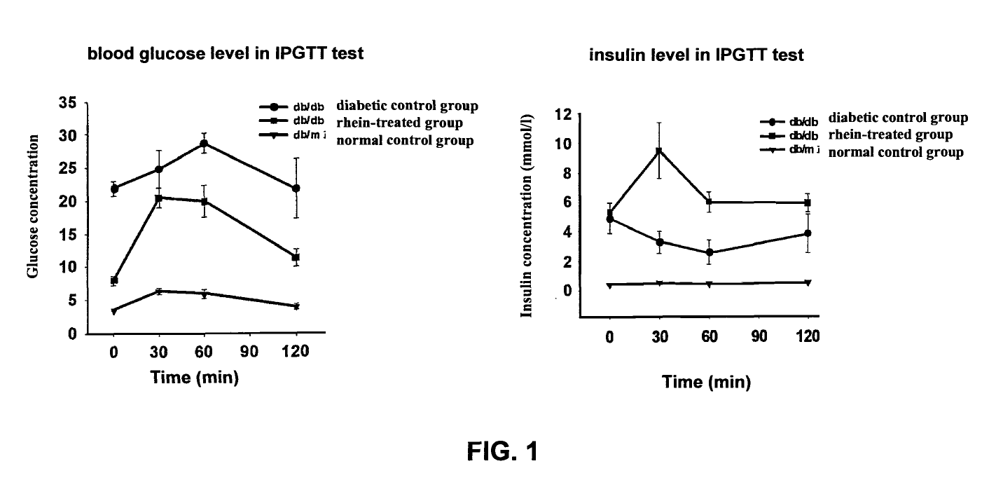

According to the experiments of the present invention, it is also shown that rhein can improve glucose tolerance. The intraperitoneal glucose tolerance test (IPGTT) is performed after treatment with rhein for 8 weeks, and the results show that the blood glucose levels in rhein-treated type 2 diabetic db/db mice are significantly lower than those of the untreated control mice at 0, 60 and 120 min after glucose loading (p<0.05) (Table 2, FIG. 1A). Furthermore, the plasma insulin levels in rhein-treated type 2 diabetic db/db mice are significantly increased at 30 and 60 min (FIG. 1B). In the rhein-treated group, the area under the curve (AUC) of blood glucose is significantly reduced as compared with that of the mice in the untreated group, the AUC of insulin is significantly increased, and specifically the AUC of insulin is most significantly increased within 0-30 min after glucose loading (Table 3). The above results suggest that the improvement of glucose tolerance by rhein is resulted from the improvement of pancreatic islet β-cell functions.

TABLE 2
The results of intraperitoneal glucose tolerance test (IPGTT) on
db/db mice after treatment with rhein for 8 weeks

| Parameters | Groups | 0 min | 30 min | 60 min | 120 min |
|---|---|---|---|---|---|
| Blood | db/m | 3.5 ± 0.2* | 6.3 ± 0.5* | 5.9 ± 0.7* | 3.9 ± 0.4* |
| glucose | db/db | 21.9 ± 1.1 | 24.8 ± 2.8 | 28.8 ± 1.6 | 21.8 ± 4.6 |
| (mmol/L) | rhein- | 7.6 ± 0.7* | 19.5 ± 1.5 | 18.3 ± 2.0* | 10.4 ± 1.1* |
| | treated | | | | |
| | db/db | | | | |
| plasma | db/m | 0.4 ± 0.1* | 0.5 ± 0.0* | 0.4 ± 0.1* | 0.5 ± 0.1* |
| insulin | db/db | 4.9 ± 1.0 | 3.3 ± 0.7 | 2.6 ± 0.8 | 3.8 ± 1.3 |
| (ug/L) | rhein- | 5.3 ± 0.7 | 9.5 ± 1.9* | 6.0 ± 0.7* | 5.9 ± 0.6 |
| | treated | | | | |
| | db/db | | | | |

Compared with db/db mice, *p < 0.05

TABLE 3
AUC of glucose and insulin in intraperitoneal glucose tolerance test
(IPGTT)

| Parameters | db/m | db/db | rhein-treated db/db |
|---|---|---|---|
| AUC-glucose | 627 ± 56* | 3023 ± 251 | 1834 ± 151* |
| AUC-insulin | 57 ± 7* | 401 ± 85 | 812 ± 80* |
| AUC_{INSO-30} | 15 ± 3* | -24 ± 10 | 63 ± 32* |

AUC_{INSO-30}: AUC of insulin within 0-30 min, compared with db/db mice, *p < 0.05

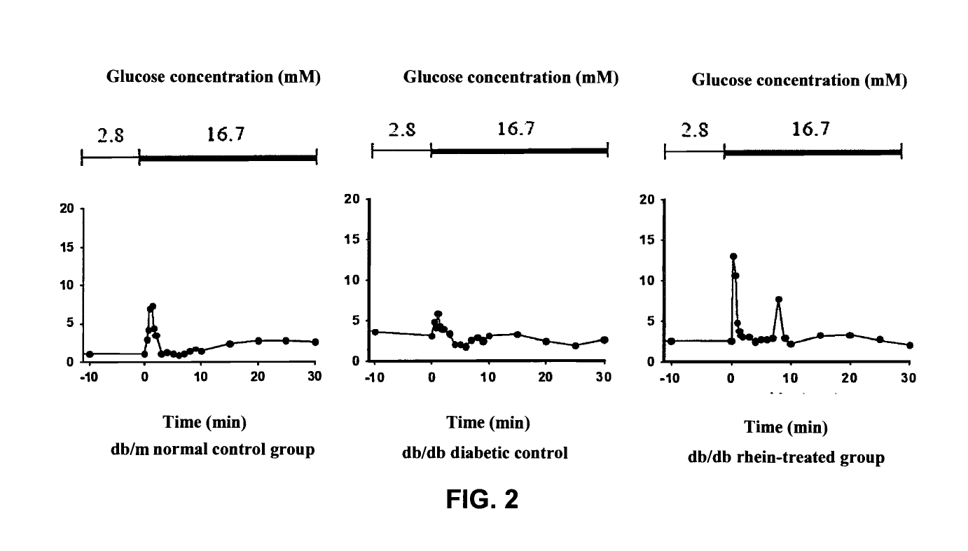

It is shown by the experiments of the present invention that rhein increases the first phase insulin secretion in type 2 diabetic db/db mice. Pancreatic islet perfusion is a golden standard for evaluating the first phase insulin secretion. The insulin-secreting function of pancreatic islet β-cells is fully evaluated in the respect of secretion phases and secretion amounts. With stimulation of high glucose of 16.7 mmol/L, the insulin level of untreated db/db mice is increased slightly, and the peak insulin level is merely 3 times as high as basal level. In contrast, the insulin level of rhein-treated mice is significantly increased after high glucose stimulation for 1 min, and is 7 times as high as basal level (FIG. 2). There is a significant difference between two groups.

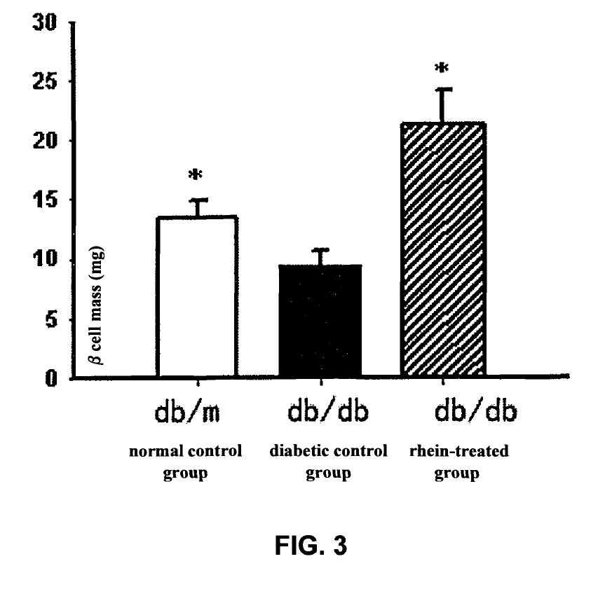

It is shown by the experiments of the present invention that rhein increases the amount of pancreatic islet β-cells. After administration for 8 weeks, the pancreatic islet β-cell amount in the untreated db/db group is considerable low, while rhein treatment significantly reduces the loss of pancreatic islet β-cells (FIG. 3). In the normal control group of db/m mice, the insulin is strongly strained and uniformly distributed in the pancreatic islets. In contrast, in the diabetic control group of db/db mice, the pancreatic islets exhibit week and sparse insulin expression, and the staining intensity is merely 50% that of the db/m control group. Compared with db/db control group, rhein treatment significantly enhances insulin expression level in the pancreatic islets.

According to the oral absorption kinetics and pharmacokinetics of rhein in rats, it is shown that after ig (intragastric gavage) administration with 35 mg/kg, 70 mg/kg or 140 mg/kg rhein, the calculated half lives are 3.22±1.21 h, 3.68±1.42 h and 4.30±1.55 h, respectively; the actually-measured peak times are 0.42±0.26, 0.50±0.27 h and 0.38±0.14 h, respectively; the peak concentrations are 37.96±12.87 μg/ml, 54.64±11.60 μg/ml and 67.17±14.62 μg/ml, respectively; and the AUCs are 69.52±9.13 μg.h/ml, 164.29±44.77 μg.h/ml and 237.75±42.81 μg.h/ml, respectively. The relationship between AUC and dosage as well as the relationship between peak concentration and dosage shows that there is a linear relationship between AUC and dosage. The three dosages exhibit the similar half lives. The above results show that in the tested dosage range, the pharmacokinetics of rhein in rats is approximately linear.

TABLE 4
Plasma rhein concentration (μg/ml) in rats after gavage administration with 35 mg/kg rhein

| Time (h) | 1 | 2 | 3 | 4 | 5 | 6 | X̅ | s |
|---|---|---|---|---|---|---|---|---|
| 0.033 | 15.34 | 19.11 | 10.10 | 10.54 | 24.21 | 9.45 | 14.79 | 5.94 |
| 0.083 | 22.07 | 44.52 | 22.33 | 16.18 | 40.94 | 19.99 | 27.67 | 11.92 |
| 0.25 | 27.43 | 45.05 | 26.00 | 26.86 | 59.77 | 24.59 | 34.95 | 14.33 |
| 0.50 | 22.01 | 22.71 | 28.87 | 26.88 | 55.32 | 22.99 | 29.80 | 12.79 |
| 0.75 | 18.92 | 18.72 | 35.43 | 35.51 | 45.07 | 15.62 | 28.21 | 12.04 |
| 1.0 | 19.13 | 19.96 | 21.75 | 27.32 | 37.07 | 15.78 | 23.50 | 7.66 |
| 2.0 | 8.25 | 4.26 | 7.88 | 7.44 | 7.45 | 15.41 | 8.45 | 3.70 |
| 3.0 | 4.03 | 4.23 | 4.39 | 4.04 | 2.99 | 4.38 | 4.01 | 0.52 |
| 4.0 | 3.98 | 4.16 | 1.70 | 3.75 | 1.31 | 4.91 | 3.30 | 1.45 |
| 6.0 | 3.08 | 2.69 | 3.08 | 2.89 | 0.57 | 0.86 | 2.19 | 1.16 |
| 8.0 | 4.99 | 0.75 | 1.94 | 2.05 | 1.07 | 0.34 | 1.86 | 1.68 |
| 12.0 | 0.58 | 0.18 | 1.13 | 1.35 | ND | 0.36 | 0.60 | 0.54 |
| 14.0 | 0.51 | ND | 0.50 | 0.56 | ND | ND | 0.35 | 0.27 |

ND: Lower than the minimal detectable concentration 0.13 μg/ml.

TABLE 5
Plasma rhein concentration (μg/ml) in rats after gavage administration with 70 mg/kg rhein

| Time (h) | 1 | 2 | 3 | 4 | 5 | 6 | x̅ | s |
|---|---|---|---|---|---|---|---|---|
| 0.033 | 34.64 | 37.28 | 24.38 | 23.12 | 14.12 | 14.57 | 24.68 | 9.74 |
| 0.083 | 30.38 | 48.63 | 34.14 | 42.68 | 26.18 | 22.27 | 34.05 | 10.01 |
| 0.25 | 38.86 | 65.89 | 62.98 | 49.02 | 24.12 | 31.05 | 45.32 | 16.99 |
| 0.50 | 50.53 | 43.37 | 44.14 | 56.14 | 33.63 | 52.37 | 46.70 | 8.05 |
| 0.75 | 30.33 | 40.26 | 34.20 | 46.37 | 24.94 | 49.16 | 37.54 | 9.41 |
| 1.0 | 30.42 | 40.86 | 29.41 | 35.89 | 20.75 | 58.65 | 36.00 | 12.99 |
| 2.0 | 13.29 | 34.33 | 26.24 | 26.80 | 15.09 | 12.71 | 21.41 | 8.95 |
| 3.0 | 13.61 | 21.97 | 27.09 | 11.54 | 16.35 | 9.87 | 16.74 | 6.62 |
| 4.0 | 7.23 | 12.76 | 19.78 | 13.32 | 14.65 | 10.51 | 13.04 | 4.20 |
| 6.0 | 4.81 | 6.74 | 18.98 | 7.55 | 3.85 | 5.39 | 7.89 | 5.59 |
| 8.0 | 6.32 | 7.32 | 11.38 | 6.09 | 2.52 | 6.32 | 6.66 | 2.84 |
| 12.0 | 2.52 | 5.62 | 6.21 | 4.94 | 1.14 | 2.79 | 3.87 | 2.00 |
| 14.0 | 0.44 | 0.40 | 0.93 | 3.56 | 1.75 | 1.91 | 1.50 | 1.19 |

TABLE 6
Plasma rhein concentration (μg/ml) in rats after gavage administration with 140 mg/kg rhein

| Time (h) | 1 | 2 | 3 | 4 | 5 | 6 | x̅ | s |
|---|---|---|---|---|---|---|---|---|
| 0.033 | 13.55 | 21.88 | 9.42 | 14.25 | 22.00 | 13.23 | 15.72 | 5.10 |
| 0.083 | 30.55 | 35.10 | 46.58 | 22.35 | 50.52 | 24.47 | 34.93 | 11.54 |
| 0.25 | 61.22 | 59.53 | 69.37 | 27.07 | 54.64 | 64.74 | 56.09 | 15.06 |
| 0.50 | 51.20 | 87.95 | 82.06 | 52.43 | 31.95 | 45.63 | 58.54 | 21.83 |
| 0.75 | 33.65 | 64.91 | 48.63 | 44.00 | 31.47 | 39.56 | 43.70 | 12.18 |
| 1.0 | 30.46 | 49.08 | 43.04 | 18.64 | 24.25 | 35.51 | 33.50 | 11.43 |
| 2.0 | 22.45 | 35.15 | 37.37 | 14.29 | 28.00 | 37.98 | 29.21 | 9.47 |
| 3.0 | 20.79 | 25.58 | 28.39 | 16.91 | 17.23 | 24.19 | 22.18 | 4.66 |
| 4.0 | 15.52 | 24.69 | 20.82 | 16.50 | 26.59 | 18.69 | 20.47 | 4.44 |
| 6.0 | 10.85 | 19.98 | 20.49 | 17.58 | 17.34 | 19.34 | 17.59 | 3.54 |
| 8.0 | 6.47 | 15.42 | 14.46 | 10.29 | 13.74 | 11.21 | 11.93 | 3.31 |
| 12.0 | 4.11 | 2.48 | 4.35 | 6.63 | 11.63 | 6.14 | 5.89 | 3.19 |
| 14.0 | 3.40 | 3.15 | 4.05 | 4.81 | 6.83 | 4.27 | 4.42 | 1.33 |

### Example 1

### Improvement Effects of Rhein on Glycuse Tolerance

Agents: Rhein dissolved in 0.1% cellulose sodium.

Administration to the experimental animals: Thirty diabetic db/db mice of 4 week old are randomly assigned to treatment group and control group. Additional fifteen normal db/m mice of 4 week old are used as normal control group. The treatment group of diabetic db/db mice is treated with rhein (120 mg/Kg, dissolved in 0.1% cellulose sodium) by gavage for continuous 8 weeks. The diabetic control group of db/db mice and the normal control group of db/m mice are administrated 0.1% cellulose sodium by gavage.

Experimental methods: The intraperitoneal glucose tolerance test (IPGTT) is carried out on the mice after administration for 8 weeks. The mice are fasted overnight, and then are administrated by i.p. injection of glucose at 0.5 g/kg body weight. Blood is collected at 0, 30, 60 and 120 min for detecting whole blood glucose level and insulin level and calculating the area under the curve (AUC) of insulin. The area under the curve of insulin during 0˜30 min (AUC_{INS0.30}) is calculated as: (insulin level at 30 min−insulin level at 0 min)×15, for evaluating the ability of early phase insulin secretion.

Experimental results: Rhein can improve glucose tolerance. The results of intraperitoneal glucose tolerance test (IPGTT) show that after treatment with rhein for 8 weeks, the blood glucose levels in rhein-treated type 2 diabetic db/db mice are significantly lower than those of the untreated control mice at 0, 60 and 120 min after glucose loading (p<0.05) (Table 2, FIG. 1A). Furthermore, the plasma insulin levels in rhein-treated type 2 diabetic db/db mice are significantly increased at 30 and 60 min (FIG. 1B). In the rhein-treated group, the area under the curve (AUC) of blood glucose is significantly reduced as compared with that of the mice in the untreated group, the AUC of insulin is significantly increased, and in particular the AUC of insulin is most significantly increased during 0-30 min after glucose loading (Table 3). The above results suggest that the improvement of glucose tolerance by rhein is resulted from the improvement of pancreatic islet β-cell functions.

### Example 2

### Effects of Rhein on the First Phase Insulin Secretion in Type 2 Diabetic db/db Mice

The agents and animals used in this example are the same as those used in example 1.

Experimental methods: After 8 weeks of administration, 5 mice are randomly selected from each group for pancreatic islet isolation and perfusion as follows. After anesthesia, the opening of common bile duct at duodenal papilla for each animal is clamped in vivo. 2 ml IV type collagenase is injected at the concentration of 1 mg/ml after performing common bile duct puncture under a stereoscopic microscope. After entering the pancreatic duct reversely to expand the pancreas, the pancreas is rapidly removed. The pancreas is placed in Hank's buffer containing 1 mg/ml collagenase and digested for 40 min to remove the collagens, and then washed under vibration for several times. Pancreatic islets are successfully isolated under microscopy. The pancreatic islets are incubated in a CO₂ incubator for 2 hour with 50 islets per group, and then the islets are placed into a specially manufactured constant-temperature perfusion equipment. The mice are perfused with 2.8 mM glucose using a Harvard micro-pump at 0.5 ml/min for glucose starvation perfusion, and after 30 min, perfusion of high glucose (16.7 Mm) is performed at 1 ml/min. The effluent liquid is collected every 20 s for first 5 mM, and then collected every 1 min. The collected effluent liquids are stored for insulin level detection via ELISA. The first phase insulin secretion and dynamic secretion level of insulin are reflected from the curve of insulin levels measured.

Experimental results: This experiment shows that rhein increases the first phase insulin secretion in type 2 diabetic db/db mice. Pancreatic islet perfusion is a golden standard for evaluating the first phase insulin secretion. The insulin-secreting function of pancreatic islet β-cells is fully evaluated in the respect of secretion phases and secretion amounts. With the stimulation of high glucose of 16.7 mmol/L, the insulin level of untreated db/db mice is increased slightly, and the peak insulin level is merely 3 times as high as basal level. In contrast, the insulin level of rhein-treated mice is significantly increased after high glucose stimulation for 1 min, and is 7 times as high as basal level (FIG. 2). There is a significant difference between two groups.

### Example 3

### Effects of Rhein on the Amount of Pancreatic Islet β-Cells

The agents and animals used in this example are the same as those used in example 1.

Experimental methods: Immunohistochemical assay. The mice are anaesthetized with phenobarbital sodium, perfused with physiological saline via heart, and fixed with 4% polyoxymethylene. The pancreas are removed, and then placed in 4% polyoxymethylene for 4-6 hours, embedded in paraffin, and sliced into a thickness of 5 um. The slices are dewaxed with xylene, rehydrated with ethanol at different gradient concentrations, and then treated with 0.3% hydrogen peroxide at room temperature for 20 min, so as to block the activity of endogenous peroxidase. The samples are treated with steams at 121° C. under high pressure for 10 min for repairing the antigen. 10% goat serum is added for blocking non-specific antigens. Rabbit-anti-mouse insulin antibody is added, and reacted at 4° C. overnight for 14 hours. Biotin-labeled goat-anti-rabbit secondary antibody is added and reacted at room temperature for 30 min. Diaminobenzidine is added for developing color. The samples are stained with hematoxylin, dehydrated and embedded.

Metrology analysis of pancreatic islets: All of the slices are observed with optical microscope E800 (Nikon, Japan), and photographed with a digital camera (Sony, Japan) connected with the microscope. The digital photographs are obtained via Axiovision 4.3 software, and analyzed with Image-Pro Plus 5.0.1. Fifteen pancreatic islet photographs are randomly selected for each mouse, and at least 50 pancreatic islet photographs are analyzed for each group. The pancreatic islet β-cell amounts are determined by analyzing the insulin-stained photographs, and calculated according the following formula: pancreatic islet β-cell amount (mg)=(the area of the pancreatic islet (β-cells/the area of the pancreas)×the mass of the pancreas (15 pancreas photographs/group). The staining intensity of insulin is determined with Scion Image B4. 0. 3 for windows (U.S.) (30 pancreatic islets/group).

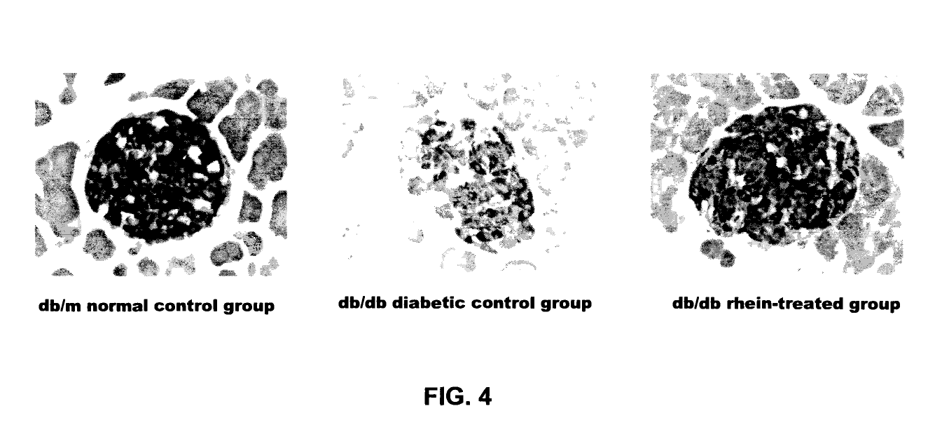

Experimental results: This experiment shows that rhein increases the amount of pancreatic islet β-cells. After administration for 8 weeks, the pancreatic islet β-cell amount in the untreated db/db group is considerable low, while rhein treatment significantly reduces the loss of pancreatic islet β-cells (FIG. 3). In the normal control group of db/m mice, the insulin is strongly strained and uniformly distributed in the pancreatic islets. In contrast, in the diabetic control group of db/db mice, the pancreatic islets exhibit week and sparse insulin expression, the staining intensity is merely 50% that of the db/m control group. Compared with db/db control group, rhein treatment significantly enhances insulin expression level in the pancreatic islets (FIG. 4).

### Example 4

### Protective Effects of Rhein on Pancreatic Islet β-Cells of Obese Rats

The agents used in this example are the same as those used in example 1.

Experimental animals: Insulin-resistant obese rats are 4-week in-bred female Wistar rats induced by feeding with high-sugar high-fat feedstuff (20% sugar, 10% lard, 2.5% cholesterol, 1% cholic acid and 66.5% normal feedstuff) for 3 weeks.

Administration to the experimental animals: Thirty insulin-resistant obese rats are randomly assigned to treatment group and control group. Additional fifteen normal Wistar rats of 7 week old are used as normal control group. The treatment group of insulin-resistant obese rats is treated with rhein (100 mg/Kg, dissolved in 0.1% cellulose sodium) by gavage for continuous 4 weeks. The insulin-resistant obese control group and the normal control group are administrated with 0.1% cellulose sodium by gavage.

Experimental results: The hyperglycemic clamp experiment shows that the glucose infusion rate (GIR) at steady-state in the rhein-treated group [(26.1±2.9) mg.kg⁻¹ min⁻¹] is significantly higher than that in the untreated obese group [(35.9±4.1) mg.kg⁻¹.min⁻¹, P<0.05] after treatment with rhein for 4 weeks (Table 1). In the normal control group, the insulin is strongly strained and uniformly distributed in the pancreatic islets. The insulin expression level in the obese control group is merely 70% that in the normal control group. Compared with the rats in the obese control group, rhein treatment significantly improves the insulin expression level in the islets.

### Example 5

### Oral Absorption Kinetics and Pharmacokinetic Parameters of Rhein in Rats

The agents used in this example are the same as those used in example 1.

Grouping of the animals and experimental procedure: Eighteen Wistar rats (male: female=1:1; body weight=180˜210 g) are randomly divided into 3 groups, 6 animal each group (male 3, female 3). The animals are fastened but supplied with free water for 10 h, and then are administrated ig with 35 mg/kg, 70 mg/kg or 140 mg/kg rhein. Blood samples are collected from carotid artery cannula at 0.033 h, 0.083 h, 0.25 h, 0.5 h, 0.75 h, 1 h, 2 h, 3 h, 4 h, 6 h, 8 h, 12 h and 14 h after administration, and centrifuged. 50 μl plasma is used for HPLC-fluorescence (FLD) analysis. The obtained plasma rhein concentration-time data are used for calculating pharmacokinetic parameters via corresponding pharmacokinetic programmes.

Experimental results: after ig administration with 35 mg/kg, 70 mg/kg or 140 mg/kg rhein, the calculated half lives are 3.22±1.21 h, 3.68±1.42 h and 4.30±1.55 h, respectively; the actually-measured peak times are 0.42±0.26, 0.50±0.27 h and 0.38±0.14 h, respectively; the peak concentrations are 37.96±12.87 μg/ml, 54.64±11.60 μg/ml and 67.17±14.62 μg/ml, respectively; and the AUCs are 69.52±9.13 μg.h/ml, 164.29±44.77 μg.h/ml and 237.75±42.81 μg.h/ml, respectively. The relationship between AUC and dosage as well as the relationship between peak concentration and dosage shows that there is a linear relationship between AUC and dosage. The three dosages exhibit the similar half lives. The above results show that in the tested dosage range, the pharmacokinetics of rhein in rats is approximately linear.

What is claimed is:

1. A method for inhibiting pancreatic islet β-cell dysfunction, comprising administering to a subject in need thereof an inhibitory effective amount of a rhein compound having the general formula (I) or a pharmaceutically acceptable salt thereof, 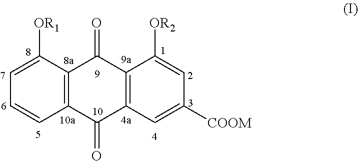
wherein M is H, alkali metal, alkaline-earth metal or organic base residue, and R₁ and R₂ are independently selected from H and acetyl.

2. The method according to claim 1, wherein both R₁ and R₂ are H, or both R₁ and R₂ are acetyl.

3. The method according to claim 1, wherein M is H or alkali metal.

4. The method according to claim 2, wherein M is H or alkali metal.

5. A method for preventing or treating a pancreatic islet β-cell dysfunction related disorder, comprising administering to a subject in need thereof a therapeutic effective amount of a rhein compound of formula (I) or a pharmaceutically acceptable salt thereof, 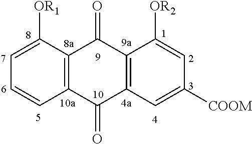
wherein M is H, alkali metal, alkaline-earth metal or organic base residue, and R₁ and R₂ are independently selected from H and acetyl.

6. The method according to claim 5, wherein both R₁ and R₂ are H, or both R₁ and R₂ are acetyl.

7. The method according to claim 5, wherein M is H or alkali metal.

8. The method according to claim 6, wherein M is H or alkali metal.

9. The method according to claim 5, wherein the disorder is selected from the group consisting of obesity and diabetes mellitus.

10. The method according to claim 9, wherein the disorder is type 2 diabetes.
